(19) United States
(12) Patent Application Publication
(10) Pub. No.: US 2013/0237504 A1
(43) Pub. Date: Sep. 12, 2013

(54) Pharmaceutical Forms and Methods for Designing the Same

(71) Applicants: Mehdi El Glaoui, Geneve (CH); Guillaume El Glaoui, Paris (FR); Antoine Ferry, Paris (FR); Anne-Emmanuelle Lugrin, Antibes Juan-les-Pins (FR); Jean-Pierre Boissel, Tassin-la-Demi-Lune (FR)

(72) Inventors: Mehdi El Glaoui, Geneve (CH); Guillaume El Glaoui, Paris (FR); Antoine Ferry, Paris (FR); Anne-Emmanuelle Lugrin, Antibes Juan-les-Pins (FR); Jean-Pierre Boissel, Tassin-la-Demi-Lune (FR)

(21) Appl. No.: 13/418,067

(22) Filed: Mar. 12, 2012

(51) Int. Cl. G06Q 50/22; A61K 31/616; A61K 31/5415; A61K 31/403

(52) U.S. Cl. 514/161; 514/412; 514/226.5; 705/2

(57) ABSTRACT

Methods for selecting active principles to be used in association within a single pharmaceutical form, the method comprising searching, selecting and analyzing published bibliographic data. The disclosure also relates computer-implemented methods for selecting active principles to be used in association within a single pharmaceutical form, comprising: selecting with a processor-implemented process at least one listing of keywords stored in memory; and submitting the keywords in a query. Further, the specification relates to pharmaceutical oral forms defined by implementing these methods, wherein at least two active principles are brought together in a leak proof water-soluble wrapping or envelop and the active principles cannot come into contact with one another.

### BACKGROUND OF THE INVENTION

1. Field of the Invention

The present invention involves the fields of methods useful to design new pharmaceutical forms, pharmaceutical forms designed by such methods as well as systems carrying out such methods.

2. Description of Related Art

Patients needing to be cured for several simultaneous diseases are classically given separate parallel and independent treatments for each disease, therefore involving as many approved medicines as the different diseases. Each particular medicine is a standalone formulation containing a specific dose of drug and excipients, and indicated for a specific target population. However, such therapeutic approaches do not allow optimized treatment. For instance, the drug dosing or the choice of excipients for each medicine may not be optimal considering the drug dosing or the choice of excipients for the other medicine(s) that is (are) taken simultaneously by the patient. In addition, the necessity of taking several medicines at the same time entail a risk of mistakes, especially when elderly patients are concerned.

### SUMMARY OF THE INVENTION

It is a first object of the present invention to provide a method for selecting active principles, e.g., generic active principles, e.g., two, three, four, five or more generic active principles, to be used in association within a single pharmaceutical form, the method comprising the steps of searching, selecting and analyzing published bibliography data for:

a) determining a therapeutic objective;

b) within the therapeutic objective, selecting and/or ranking a first series of active principles and/or class(es) of active principles;

c) selecting related data susceptible to lead to a rationale of association with another series of active principles and/or class(es) of active principles; and

d) refining selection of candidate active principles and ending up with an association of two, three, four, five or more active principles.

Step a) may include collecting data related to significant prevalence of a pathological or pluripathological situation; and/or Public Health Impact of the disease, severity and its consequences on patients' quality of life; and/or availability of efficient pharmaceutical therapies, and/or compliance issues due to the context, essentially polymedication; and/or clearly identifiable therapeutic clinical objective which is meaningful at the individual (patient) and collective (public health) levels.

Step b) may include collecting data related to the presence of a sub-classification; and/or efficacy criteria; and/or dose-effect relationship; and/or specific benefits of some active principles or sub-class; presence of a need of co-therapy. The selection of the active principles is defined with consideration of the therapeutic efficacy in the therapeutic objective; and/or the risk of serious undesirable effects of each candidate active principle; and/or the frequency of undesirable effects of each candidate active principle; and/or the compatibility of each candidate active principle with other candidate active principle(s) of the association. The risk of undesirable effects or serious side effects may be evaluated by searching and analyzing the frequency of occurrence of said effects in available compliance studies and drug monitoring reports, and meta-analysis of randomized clinical trials.

Step c) may include selecting a therapeutic indication for a clinical situation requiring a simultaneous prescription of two or more candidate active principles.

Step d) may include refining therapeutic indication; and/or considering the therapeutic efficacy and/or benefit of each candidate active principle; and/or refining target population; and/or considering interactions between candidate active principles and/or pharmaceutical forms; and/or refining dosage regimen. The step of refining therapeutic indication includes searching and analyzing information related to approved indication for each candidate active principle of the association; and/or clinically tested formulations for each candidate active principle of the association; and/or clinical situation for which complementarity of the candidate active principles benefits the most; and/or clinical situation with strong compliance problem; and/or Recommendations for Good Practices (RGP). The step of considering the therapeutic efficacy and/or benefit of each candidate active principle is searched and has to be found as evidenced and quantitatively evaluated in randomized clinical trials and/or in appropriate meta-analyses with sufficient levels of proof. The step of refining target population includes analyzing randomized clinical trials in order to identify the type of patients that may benefit of the association of candidate active principles; and/or discarding the patient populations that may not benefit from associations of candidate active principles. The step of refining dosage regimen includes searching and analyzing one, two, three, four, five or all the following criteria:
pharmacokinetics, in order to determine the minimum daily dosage for the association; and/or
dose-effect relationship on the therapeutic indication as determined during randomized clinical trials, or trials considering an intermediate criterion; and/or
descriptions of Summary of Product Characteristics (SPC); and/or
Recommendations for Good Practices; and/or
consumption statistics.

According to an embodiment of the invention, the database search is performed through binary clinical terms, integrative clinical terms, and/or success/failure criterion, and is preferably directed for a need of a co-therapy.

According to an embodiment of the invention, at least one of the active principles is a generic active principle. More preferably, all of the active principles are generic active principles.

It is a further object of the present invention to provide a computer-implemented method for selecting active principles to be used in association with a single pharmaceutical form, comprising steps of: selecting with a processor-implemented process at least one listing of keywords stored in memory; and submitting the keywords in a query.

It is a further object of the present invention to provide a pharmaceutical oral form defined by implementing the method of the invention, wherein at least two active principles are brought together in a leak proof water-soluble wrapping or envelop and the active principles cannot come into contact with one another.

### BRIEF DESCRIPTION OF THE DRAWINGS

The foregoing summary, as well as the following detailed description of the invention, will be better understood when read in conjunction with the appended drawings. For the purpose of illustrating the invention, certain preferred embodiments are shown in the drawings. It should be understood, however, that the invention is not limited to the precise arrangements and instrumentalities shown.

FIG. 1 is a flow chart illustrating the method according to the present invention.

FIG. 2 is a schematic view of a first embodiment of a pharmaceutical form according to the present invention showing a wrapping (or coating) 1 containing two medicines 2 and 3, each one containing an active principle, and a separating sheet or wall 4.

FIG. 3 is a schematic view of a second embodiment of a pharmaceutical form according to the present invention showing a wrapping (or coating) 1 containing three medicines 2, 3, 5, each one containing an active principle, and a separating sheet or wall 4.

FIG. 4 is a schematic view of a third embodiment of a pharmaceutical form according to the present invention showing a capsule made of two half-capsules 1a and 1b, each one containing a medicine (2, 3) containing an active principle, and a separating sheet or wall 4.

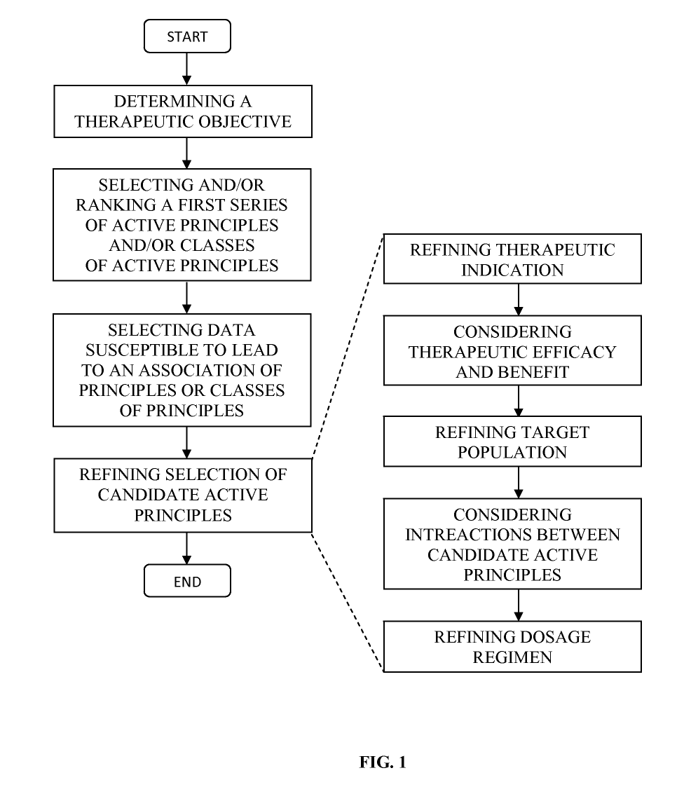

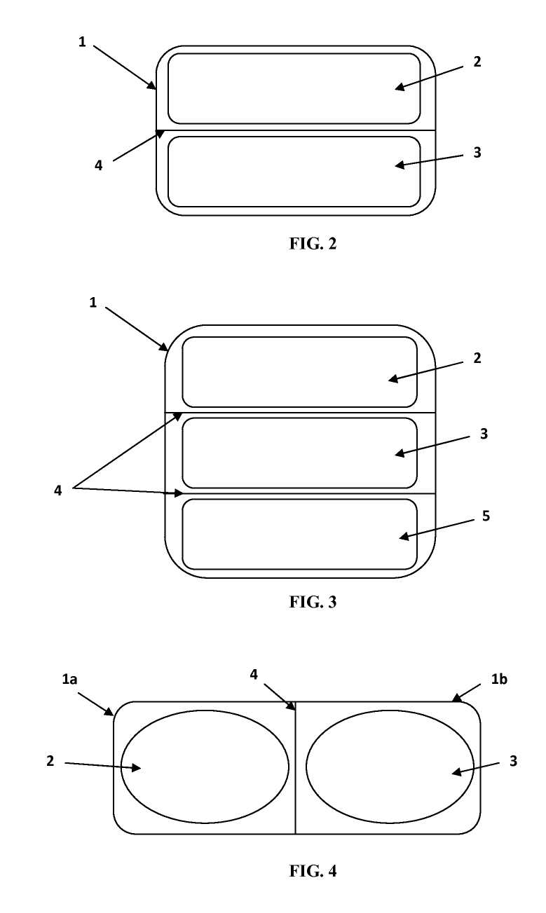

### DEFINITIONS

Unless otherwise indicated, all terms used herein have the same meaning as they would to one skilled in the art. The practice of the present invention will employ conventional techniques of drug selection and computer technology, which are within the knowledge of those of skill of the art.

In describing the present invention, the following terms are employed and are intended to be defined as indicated below.

The term “candidate active principle” refers to a drug which is selectable to be used in association within a pharmaceutical form according to the present invention.

The term “class effect” refers to drugs that may be grouped together as a class involving some or all of the following criteria: similar chemical structure, mechanism of action or pharmacological effects.

The term “clinically tested formulations” refers to the clinically tested physical form in which a drug is produced and dispensed, such as a tablet, a capsule, or an oral or injectable solution.

The term “compatibility”, when referring to a candidate active principle, is to be understood as opposed to the term “incompatibility” that refers to a situation where one or more active principles exhibit chemical, pharmacological and/or pharmaceutical incompatibilities.

The term “compliance study” refers to a study measuring how well subjects have taken the study medications in the prescribed frequency and/or followed protocol required tests and visit schedules.

The term “consumption statistics” refers to statistics relating to the amounts of medicines distributed legally in a country for medical purposes to those institutions and programs that are licensed to dispense to patients, such as hospitals, nursing homes, pharmacies, hospices and palliative care programs. In international drug control terminology, consumption does not refer to the amounts dispensed to or used by patients, but rather amounts distributed to the retail level.

The term “co-therapy” refers to the simultaneous therapy by two, three, four, five or more drugs of two, three, four, five or more different pathologies.

The term “dose-effect relationship” refers to the link between the total amount of a compound administered, or absorbed by a system and the magnitude of a specific, continuously graded change affecting it.

The terms “drug” and “active principle” are used interchangeably herein to mean a chemical substance used in the treatment, cure, prevention, or diagnosis of disease, or used to otherwise enhance physical or mental well-being.

The term “meta-analysis” refers to a quantitative statistical analysis of several separate but similar experiments or studies in order to test the pooled data for statistical significance.

The term “generic active principle” refers to an active principle which is the same as a brand name active principle in dosage, safety, strength, how it is taken, quality, performance, and intended use.

The term “pharmacokinetics” refers to the study of processes, in a living organism, of absorption, distribution, metabolism, and excretion of a drug or vaccine.

The term “pharmacodynamics” refers to the study of reactions between drugs and living structures, including the physiological responses to pharmacological, biochemical, physiological, and therapeutic agents.

The term “randomized clinical trials” refers to medical research studies in which one or more groups are formed by random assignment to treatments and controls therefore allowing groups to be more equivalent when comparing the effects of treatment.

The term “scientific data source” refers to a bibliographic database containing references to published literature in biological and biomedical sciences and searchable by keywords.

The term “serious side effect” refers to any untoward medical occurrence that, at any dose, results in death, is life threatening, requires inpatient hospitalization or prolongation of existing hospitalization, results in persistent or significant disability/incapacity, or is a congenital anomaly/birth defect.

The term “Summary of Product Characteristics (SPC)” refers to the basis of information for health professionals on how to use a medicinal product safely and effectively.

The term “target population” refers to a particular group of patients that is identified as the intended recipient of a treatment.

The term “therapeutic efficacy” refers to the capacity of a drug or treatment to produce beneficial effects on the course or duration of a disease at the dose tested and against the illness for which it is designed.

The term “therapeutic indication” refers to a symptom or particular circumstance that indicates the advisability or necessity of a specific medical treatment or procedure.

The term “undesirable effects” refers to any undesired actions or effects of a drug or treatment.

### DETAILED DESCRIPTION OF THE INVENTION

A main objective of the present invention is to provide a method for designing a pharmaceutical form for optimized and cost-saving treatment of patients in need of a therapy involving multiple, e.g. two, three, four, five or more drugs.

The invention also relates to a method for designing a pharmaceutical form involving multiple drugs, which is performed by searching, selecting and analyzing published bibliographic data relating to drugs, their therapeutic use, their posology, the circumstances under which they may be prescribed, their target population and/or their possible risks, interactions and/or serious side effects.

Another object of this invention is a method for selecting active principles, e.g., generic active principles, e.g., two, three, four, five or more generic active principles, to be used in association within a single pharmaceutical form.

The methods of the invention imply accessing at least one scientific data source that stores scientific data relating to active principles. Various appropriate scientific data sources may be easily selected and accessed for running a particular search in the context of the present invention.

In an embodiment, scientific data sources may be meta-analysis (MA) of randomized clinical trials (RCT) which provide the highest levels of proof in terms of patient benefit according to the majority of current classification systems. Further preferred scientific data sources are general reviews, compliance studies and drug monitoring articles. Further scientific data sources may be PubMed databases. The database Medline (http://www.nlm.nih.gov/databases/databases_medline.html) may for example be used via the PubMed interface (http://www.ncbi.nlm.nih.gov/pubmed/).

Lists of bibliographic references available from further scientific data sources may be also selected, provided that they are based on explicit levels of proof. Such further data sources are for example the reports of the British “Health Technology Assessment” (National Institute for Health Research), the Canadian Coordinating Office for Health Technology Assessment, or the “Clinical Evidence” (British Medical Journal Publishing Group).

Database search according to the invention is preferably performed via a computerized approach using keyword-based search(es) looking for words anywhere in the scientific data source(s), or using search options combining search elements, group terms, or selecting indexes or fields to be searched within the data source(s).

Database search according to the invention may use syntax search favoring sensitivity of the searches over their specificity, such as for example by comparing MeSH (Medical Subject Headings) descriptors, MeSH being the NLM (U.S. National Library of Medicine) controlled vocabulary thesaurus used for indexing articles for PubMed. When the PubMed database is used, a search may be performed by interrogating the “Clinical Queries” (page http://www.ncbi.nlm.nih.gov/pubmed/clinical), the “Therapy” entry which filters the randomized control trials (RCT) and the meta-analysis of RCT.

Further searches may be performed on compliance data related to the selected pathological domain, or for the contemplated associated therapies.

The method of the invention includes assessing the benefits and risks of associating at least two active principles.

Assessing the benefits and risks of associating at least two active principles includes assessing the benefits/risks ratio of associating the therapeutic classes to which said active principles belong, for a selected therapeutic indication. Said benefits and risks are assessed, in a first phase, on the basis of analysis of morbi-mortality data and/or clinical criteria. In a second phase, reviewing of appropriate pivotal randomized clinical trials (RCT) aims at confirming a favorable benefits/risks ratio, further analyzing the pharmacological properties of the involved active principles, further assessing the risks of drug interactions and selecting the appropriate doses for each active principle encompassed in the pharmaceutical form of the invention.

Various appropriate scientific data sources may be easily selected and accessed for running a particular search in the context of the present invention.

Further searches may be performed on compliance data related to the selected pathological domain, or for the contemplated associated therapies.

In an embodiment, said bibliographic search may be performed according to a systematic and multimodal protocol in order to minimize the risk of false negative results.

In an embodiment of the invention, the method comprises searching, selecting and analyzing published bibliographic data for:
a) determining a therapeutic objective,
b) within the therapeutic objective, selecting and/or ranking a first series of active principles and/or class(es) of active principles,
c) selecting related data susceptible to lead to a rationale of association with another series of active principles and/or class(es) of active principles, and
d) refining selection of candidate active principles and ending up with an association of two, three, four, five or more drugs;

In a first embodiment of the invention, step a)—which is determining a therapeutic objective—may include collecting data related to:
significant prevalence of a pathological or pluripathological situation; and/or
public health impact of the disease, as a result of its prevalence, severity and its consequences on patients' quality of life; and/or
availability of efficient pharmaceutical therapies, and/or
compliance issues due to the context, essentially polymedication; and/or
clearly identifiable therapeutic clinical objective which is meaningful at the individual (patient) and collective (public health) levels.

The therapeutic objective may be found as a result of a bibliographic search in at least one scientific data source that stores scientific data relating to active principles by keywords addressing the points above.

In a second embodiment of the invention, step b)—which is selecting and/or ranking a first series of active principles and/or class(es) of active principles—may include collecting data related to:
presence of a sub-classification; and/or
efficacy criteria; and/or
dose-effect relationship; and/or
specific benefits of some active principles or sub-class; and/or
presence of a need of co-therapy (for example, to alleviate side effects).

In an embodiment, the selection of active principles may be defined with consideration of one or more, e.g., two, three or all, of the following criteria:
therapeutic efficacy in the therapeutic objective;
risk of serious undesirable effects of each candidate active principle;
frequency of undesirable effects of each candidate active principle;
compatibility of each candidate active principle with other candidate active principle(s) of the association.

According to a first variation of this embodiment, at least one of the ranked active principles is a generic active principle; according to a second variation of this embodiment all ranked active principles are generic active principles.

In an embodiment, the method of the invention may be performed through a database search involving binary clinical terms, if possible integrative clinical terms, i.e., summarizing the benefit and the risk for the patient and the collectivity, as for example the success/failure criterion.

For example, considering that the therapeutic objective is the relief of a symptom, such as pain, the active principle may be selected only if the database search indicates that when the active principle is used, the removal of said symptom occurs.

In an embodiment, the method of the invention may be performed through a database search for a need of co-therapy.

In case of a class effect characterized by, e.g., a single mechanism of action and absence of heterogeneity at the meta-analysis according to a fixed effect model on the elected therapeutic effect, the active principle fulfilling at best the preceding criterion is selected.

In absence of such class effect, the first criterion “therapeutic efficacy of each candidate active principle,” is replaced by “candidate active principle with the best therapeutic efficacy.”

When selecting active principles, the risk of undesirable effects or serious side effects may be evaluated by, e.g., considering available compliance studies and drug monitoring, and the frequency of occurrence of said effects may be evaluated by, e.g., meta-analysis in randomized clinical trials.

The risk of undesirable effects or serious side effects is evaluated by searching and analyzing the frequency of occurrence of said effects in available compliance studies and drug monitoring reports, and meta-analysis of randomized clinical trials.

In a third embodiment of the invention, step c)—which is selecting related data susceptible to lead to a rationale of association with another series of active principles or class of active principles—may include selecting a therapeutic indication for a clinical situation requiring a simultaneous prescription of two or more candidate active principles;

In a fourth embodiment of the invention, step d)—which is refining selection of candidate active principles and ending up with an association two, three, four, five or more drugs—may include one, two, three, four or all of these following steps:
refining therapeutic indication;
considering the therapeutic efficacy and/or benefit of each candidate active principle;
refining target population;
considering interactions between candidate active principles and/or pharmaceutical forms;
refining dosage regimen.

“Refining therapeutic indication” may be performed by searching in the databases one, two, three, four or all the following criteria:
approved indication for each candidate active principle of the association; and/or
clinically tested formulations for each candidate active principle of the association; and/or
clinical situation for which complementarity of the candidate active principles benefits the most; and/or
clinical situation with strong compliance problem; and/or
Recommendations for Good Practices (RGP).

According to an embodiment of the invention, “considering the therapeutic efficacy and/or benefit of each candidate active principle”, in view of the selected therapeutic indication, is searched and has to be found as evidenced and quantitatively evaluated in randomized clinical trials and/or in appropriate meta-analyses with sufficient levels of proof. The benefit may be considered “evidenced and quantitatively evaluated” even in absence of double-blind studies if evaluation of the elected criterion has been performed blind.

The target population of the step “refining target population” may be defined by, e.g., analyzing randomized clinical trials in order to identify the type of patients that may benefit of the association of candidate active principles. Individual patient data analysis may also be used. In an embodiment, the method of the invention includes discarding the patient populations that may not benefit from particular associations of candidate active principles.

The method of the invention may further comprise “considering interactions between candidate active principles and/or pharmaceutical forms”, which may be performed in various ways depending on whether the reason for a particular association relates to mechanistic, pharmacokinetics or pharmacodynamics. Interactions are avoided when mechanistic is concerned. In the two other cases, the interactions validate a particular association, and are therefore as much documented as possible.

The method of the invention may further comprise the step of “dosage regimen”, which defines doses for each candidate active principle. This step may consider one, two, three, four or all the following criteria:
pharmacokinetics, in order to determine the minimum daily dosage for the association; and/or
dose-effect relationship on the therapeutic indication as determined during randomized clinical trials, or trials considering an intermediate criterion; and/or
descriptions of Summary of Product Characteristics (SPC); and/or
Recommendations for Good Practices; and/or
consumption statistics.

The step of defining doses for each candidate active principles may further comprise considering pharmacodynamics, particularly in case of delayed effect or hysteresis. In case of discrepancy between the various data sources, a compromise is sought by hierarchizing such sources according to the preceding criteria.

According to the invention, first, second, third and/or fourth embodiment herein described, may be combined in the method of the invention.

This invention also relates to the pharmaceutical form obtained through the method herein described.

In an embodiment, the pharmaceutical form designed according to the method of the invention is a unit form or a single pharmaceutical form comprising two, three, four, five or more active principles. In a preferred embodiment, the pharmaceutical form is an oral form.

An advantage of this invention is that, according to the method of the invention, the association may gain the benefit of an abbreviated registration process with Medicine Regulatory Agencies.

The present invention may allow the collectivity and the patients to reduce overall therapeutic costs by substituting multiple prescriptions. This cost-saving relies on two main aspects: a reduced cost due to the prescription of a single pharmaceutical form instead of separate pharmaceutical forms, and a better patient compliance.

This pharmaceutical form is easy to manufacture while ensuring bioavailability of each of the associated medicines. It is designed to ensure very precise dosages and posology for patients.

The pharmaceutical form of the invention contains two, three, four, five or more medicines, each one containing an active principle, which on the one hand may be brought together in a leak proof water-soluble wrapping or envelop, and on the other hand may be separated so that the active principles of the associated medicines cannot come into contact with one another. The medicines may be separated by, e.g., a sheet which extends inside the wrapping and is made of pharmaceutically acceptable material. The wrapping and the sheet or walls are in contact in order to provide continuity of separation and protection.

By “water-soluble wrapping” is meant a wrapping that is dissolvable in an aqueous medium, at the pH of the mouth, at the pH of the stomach or at the pH of the intestine track.

After dissolution of the wrapping, the various galenical formulations or medicines are released into a body cavity in their original form, as if each of the medicines had been administered separately from the other(s).

The dosages of the medicines so associated or combined are coordinated, meaning that the dosages of the associated medicines are optimized according to an optimum therapeutic effectiveness in a single administration.

The wrapping may be of various forms. A first embodiment is of the soft capsule type and is obtained from two half-capsules of gelatin or an analogous material, which are sealed together around the combined medicines, e.g. with separation by a separating sheet or wall (or more depending on the number of medicines), the latter being, e.g., made from the same material as the capsule, for example of gelatin or the like.

In a second embodiment, the wrapping is a matrix of rice paper or analogous material, especially a matrix formed by one part, for example an oblong part, having at least two compartments, each of which receives a medicine, and one part forming a cover.

In a third embodiment, the wrapping is an inclusion matrix, the medicines being included in that matrix. The method may consist in bringing the medicines together in a mould into which a liquid binder is poured in an appropriate manner, optionally in two phases, which the binder, after curing, will form a matrix that is water-soluble under the appropriate pH conditions. The matrix may especially be made of gelatin or the like or alternatively of a polymeric material such as a polyethylene glycol, for example PEG 6000.

In a fourth embodiment, the wrapping is formed by two sheets of water-soluble material which are applied so as to surround the associated medicines. They are then sealed to one another.

In a fifth embodiment, the wrapping is a film coating present over the entire outer surface of the tablets and brought together by bonding.

In a sixth embodiment, the pharmaceutical form of the invention is as defined in international patent application WO2009092819, herein incorporated by reference.

This invention also relates to a computer-implemented method for selecting active principles to be used in association within a single pharmaceutical form, comprising steps of: selecting with a processor-implemented process at least one listing of keywords stored in memory; and submitting the keywords in a query.

Keywords related to identification of a therapeutic objective may be also related to:
significant prevalence of a pathological or pluripathological situation; and/or
public health impact of the disease, as a result of its prevalence, severity and its consequences on patients' quality of life; and/or
availability of efficient pharmaceutical therapies, and/or
compliance issues due to the context, essentially polymedication; and/or
clearly identifiable therapeutic clinical objective which is meaningful at the individual (patient) and collective (public health) levels.

### EXAMPLES

### Example 1

### Rationale for Associating in a Single Pharmaceutical Form an NSAID and PPIs (Proton-Pump Inhibitors)

As one of the most frequently prescribed class of agents [anti-inflammatory, antipyretic and analgesic effects], a search leads to select NSAID (non-steroidal anti-inflammatory drug).

In the case of NSAID, the data to collect are related to the study of:
their classification towards their selectivity to COX1 and COX2,
the criteria underlying their efficacy,
their dose-effect relationship,
their benefits.

All these studies show a class effect: all NSAID lead to comparable effects at the recommended dosages. All NSAID also lead to comparable side-effect: gastro-intestinal troubles.

The method of the invention thus points out the usefulness of a single pharmaceutical form of a non-steroidal anti-inflammatory compound and a gastroprotectant, such as for example PPIs.

### Example 2

### An Example of a Keyword-Based Search for a Ramipril-Simvastatine Combination

Keywords related to selecting or ranking a first series of active principles or class(es) of active principles, may include for example:
(Angiotensin Converting Enzyme Inhibitors) AND (mortality) AND ((cardiac failure) OR (sudden death) OR (unstable coronary insufficiency)) AND ((randomized controlled trial[Publication Type] OR (randomized[Title/Abstract] AND controlled[Title/Abstract] AND trial[Title/Abstract]))
(ramipril) AND (randomized controlled trial[Publication Type] OR (randomized [Title/Abstract] AND controlled[Title/Abstract] AND trial[Title/Abstract]))
(ramipril) AND systematic[sb]
(simvastatin) AND (randomized controlled trial[Publication Type] OR (randomized [Title/Abstract] AND controlled[Title/Abstract] AND trial[Title/Abstract]))
(ramipril) AND AND (primary prevention)
(ramipril) AND (simvastatin) AND (adverse events) AND ((randomized controlled trial[Publication Type] OR (randomized[Title/Abstract] AND controlled[Title/Abstract] AND trial[Title/Abstract]))

### Example 3

### Results

Examples of particular active principle associations according to the present invention illustrate its utility in various clinical fields.

A. Type 2 Diabetes

TABLE 1

| Indication | Active principles associations |
|---|---|
| Glycemic control, renal protection | metformine + converting enzyme |
| | inhibitor |
| Glycemic control, tensional | anticalcic + metformine + |
| objectives of the diabetic patient | converting enzyme inhibitor |
| CVR of the diabetic patient | converting enzyme inhibitor + |
| | metformine + statin |

B. Arterial Hypertension of a High Risk Subject

TABLE 2

| Indication | Active principles associations |
|---|---|
| Treatment of arterial pressure and | beta-blocker + converting enzyme |
| global cardiovascular risk (CVR) | inhibitor + statin |
| Cardiac insufficiency on arterial | beta-blocker + converting enzyme |
| hypertension (AHT) | inhibitor + thiazidic diuretic |

C. Secondary Coronary Prevention

TABLE 3

| Indication | Active principles associations |
|---|---|
| patient with proven | acetylsalicylic acid + beta-blocker + |
| coronary event | statin |
| | acetylsalicylic acid + converting enzyme |
| | inhibitor + statin |
| | acetylsalicylic acid + beta-blocker + |
| | converting enzyme inhibitor |
| | acetylsalicylic acid + beta-blocker, or |
| | acetylsalicylic acid + statin |

D. Cerebrovascular Accident (CVA) and Peripheral Arterial Disease (PAD)

TABLE 4

| Indication | Active principles associations |
|---|---|
| reduction of major cardiovascular events | acetylsalicylic acid + statin |

E. Ischemic Cardio Pathology

TABLE 5

| Indication | Active principles associations |
|---|---|
| without stent, or after | acetylsalicylic acid + beta-blocker + statin |
| 1 year under clopidogrel, | |
| or in case of associated | |
| ventricular dysfunction | |
| with sent, the first year | acetylsalicylic acid + clopidogrel + statin |
| | (if no clopidogrel-statin interaction after |
| | checking) |
| in case of associated | amiodarone + beta-blocker + converting |
| rhythm disorders | enzyme inhibitor |
| | amiodarone + acetylsalicylic acid + |
| | beta-blocker |

F. Anti-Inflammatory Treatment

TABLE 6

| Indication | Active principles associations |
|---|---|
| Anti-inflammatory treatment | NSAID (non-steroidal anti-inflammatory |
| of patients at risk of | disease) + PPI (proton pump inhibitor) |
| gastroduodenal lesions | |

NSAID components are for example found in: naproxen, ketoprofen, diclofenac or ibuprofen. PPIs are for example found in: omeprazole, pantoprazole or lansoprazole.

TABLE 7

| Indication | Active principles associations |
|---|---|
| Inflammation of osteoarthritis and | Meloxicam + omeprazole |
| rheumatoid arthritis | |

A dosage for this association may be 15 mg for meloxicam and 20 mg for omeprazole.

G. Cardiac Insufficiency

TABLE 8

| Indication | Active principles associations |
|---|---|
| Cardiac insufficiency on | beta-blocker + converting enzyme inhibitor + |
| AHT | diuretic thiazidic |
| Cardiac insufficiency on | acetylsalicylic acid + betablocker + statin |
| ischemic cardiopathy | |
| | beta-blocker + converting enzyme inhibitor + |
| | statin |

### REFERENCES

Hippisley-Cox, J. and C. Coupland (2005). “Effect of combinations of drugs on all-cause mortality in patients with ischaemic heart disease: nested case-control analysis.” BMJ 330 (7499): 1059-1063.
Yusuf, S., P. Pais, et al. (2009). “Effects of a polypill (polycap) on risk factors in middle-aged individuals without cardiovascular disease (tips): a phase ii, double-blind, randomised trial.” Lancet 373(9672): 1341-1351.

1. A method for selecting active principles to be used in association within a single pharmaceutical form, the method comprising:
searching, selecting and analyzing published bibliographic data for:
determining a therapeutic objective;
within the therapeutic objective, selecting and/or ranking a first series of active principles and/or class(es) of active principles;
selecting related data susceptible to lead to a rationale of association with another series of active principles or class(es) of active principles; and
refining selection of candidate active principles and obtaining an association of at least two, three, four, five or more active principles.

2. The method of claim 1, further comprising obtaining an association of at least three active principles.

3. The method of claim 2, further comprising obtaining an association of at least four active principles.

4. The method of claim 3, further comprising obtaining an association of at least five active principles.

5. The method of claim 1, further comprising collecting data related to significant prevalence of a pathological or pluripathological situation; and/or public health impact of the disease, severity and its consequences on patients' quality of life; and/or availability of efficient pharmaceutical therapies; and/or compliance issues due to the context, essentially polymedication; and/or clearly identifiable therapeutic clinical objective which is meaningful at the individual (patient) and collective (public health) levels.

6. The method of claim 1, further comprising collecting data related to the presence of a sub-classification; and/or efficacy criteria; and/or dose-effect relationship; and/or specific benefits of some active principles or sub-class; and/or presence of a need of co-therapy.

7. The method of claim 1, wherein selection of the active principles is defined with consideration of the therapeutic efficacy in the therapeutic objective; and/or the risk of serious undesirable effects of each candidate active principle; and/or the frequency of undesirable effects of each candidate active principle; and/or the compatibility of each candidate active principle with other candidate active principle(s) of the association.

8. The method of claim 7, wherein the risk of undesirable effects or serious side effects is evaluated by searching and analyzing the frequency of occurrence of said effects in available compliance studies and drug monitoring reports, and meta-analysis of randomized clinical trials.

9. The method of claim 1, further comprising selecting a therapeutic indication for a clinical situation requiring a simultaneous prescription of two or more candidate active principles.

10. The method of claim 1, further comprising:
refining therapeutic indication; and/or
considering the therapeutic efficacy and/or benefit of each candidate active principle;
and/or
refining target population; and/or
considering interactions between candidate active principles and/or pharmaceutical forms; and/or
refining dosage regimen.

11. The method of claim 10, wherein refining therapeutic indication includes searching and analyzing information related to approved indication for each candidate active principle of the association; and/or clinically tested formulations for each candidate active principle of the association; and/or clinical situation for which complementarity of the candidate active principles benefits the most; and/or clinical situation with strong compliance problem; and/or Recommendations for Good Practices (RGP).

12. The method of claim 10, wherein considering the therapeutic efficacy and/or benefit of each candidate active principle is searched and has to be found as evidenced and quantitatively evaluated in randomized clinical trials and/or in appropriate meta-analyses with sufficient levels of proof.

13. The method of claim 10, wherein refining target population includes analyzing randomized clinical trials in order to identify the type of patients that may benefit from the association of candidate active principles; and/or discarding the patient populations that may not benefit from associations of candidate active principles.

14. The method of claim 10, wherein refining dosage regimen includes searching and analyzing one, two, three, four, five or all the following criteria:
pharmacokinetics, in order to determine the minimum daily dosage for the association; and/or
dose-effect relationship on the therapeutic indication as determined during randomized clinical trials, or trials considering an intermediate criterion; and/or
descriptions of Summary of Product Characteristics (SPC); and/or
Recommendations for Good Practices; and/or
consumption statistics.

15. The method of claim 1, wherein the database search is performed through binary clinical terms, integrative clinical terms, and/or success/failure criterion.

16. The method of claim 1, wherein the database search is directed to a search for the need of a co-therapy.

17. The method of claim 1, wherein at least one of the active principles is a generic active principle.

18. The method of claim 1, wherein each of the at least two or more active principles is a generic active principle.

19. A pharmaceutical oral form defined by implementing the method of claim 1, wherein at least two active principles are brought together in a leak proof water-soluble wrapping or envelop and the active principles cannot come into contact with one another.

20. A computer-implemented method for selecting active principles to be used in association within a single pharmaceutical form, comprising:
selecting with a processor-implemented process at least one listing of keywords stored in memory; and
submitting the keywords in a query.

21. A pharmaceutical oral form defined by implementing the method of claim 20, wherein at least two active principles are brought together in a leak proof water-soluble wrapping or envelop and the active principles cannot come into contact with one another.
